(19) United States
(12) Patent Application Publication
(10) Pub. No.: US 2009/0054353 A1
(43) Pub. Date: Feb. 26, 2009

(54) MANNOSYL-1 PHOSPHATES, PREPARATION METHOD AND THERAPEUTIC USE, IN PARTICULAR AGAINST THE CDG-IA SYNDROME

(76) Inventors: Christine Gravier-Pelletier, Mennecy (FR); Renaud Hardre, Paris (FR); Amira Khaled, Paris (FR); Yves Le Merrer, Bonneuil-sur-Marne (FR)

Correspondence Address: OSTROLENK FABER GERB & SOFFEN, 1180 AVENUE OF THE AMERICAS, NEW YORK, NY, 100368403, US

(21) Appl. No.: 12/279,876

(22) PCT Filed: Feb. 27, 2007
(86) PCT No.: PCT/FR07/00332

§ 371 Date: Oct. 2, 2008

(30) Foreign Application Priority Data
Feb. 24, 2006 (FR) 0601646

(51) Int. Cl. A61K 31/70; C07H 3/02; A61P 25/00

(52) U.S. Cl. 514/25; 536/17.1

(57) ABSTRACT

The present invention relates, as medicaments, to the α-D-mannopyranosyl-1 phosphate derivatives of formulae I, II and III:

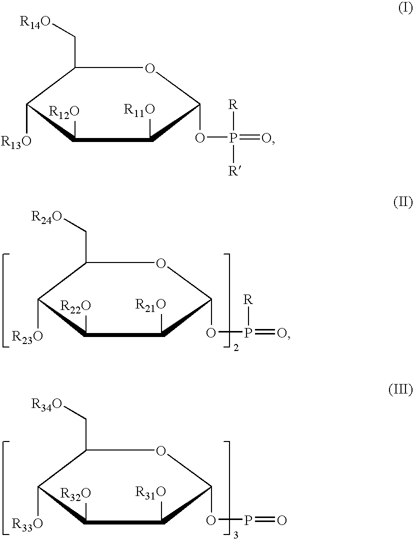

(where each group R₁₁ to R₁₄, R₂₁ to R₂₄, R₃₁ to R₃₄ is H or an OH-protective group, and R and R′ are defined as indicated in the description), which can be used as cellular sources of Man-1 P, against the CDG-I syndrome and in particular the CDG-Ia syndrome.

The invention also relates to these derivatives as industrial products and to their method of preparation.

### FIELD OF THE INVENTION

The present invention relates to a novel technical solution for the treatment of type I and more particularly type Ia CDG (congenital disorders of glycosylation) syndrome. According to the invention, this novel solution involves mannosyl-1 phosphate derivatives, namely mono-(mannopyranosyl-1), di(mannopyranosyl-1) and tri(mannopyranosyl-1) phosphates whose formulae are given below. Briefly, the invention relates more specifically to:

the mono(mannopyranosyl-1) phosphates of formula I, the di(manno-pyranosyl-1) phosphates of formula II and the tri(mannopyranosyl-1) phosphates of formula III, as medicaments which can be used against the CDG-I syndrome and more particularly against the CDG-Ia syndrome,

the therapeutic use of said mono(mannopyranosyl-1) phosphates of formula I, di(mannopyranosyl-1) phosphates of formula II and tri(mannopyranosyl-1) phosphates of formula III, in the treatment of the CDG-1 syndrome and more particularly the CDG-Ia syndrome,

the mono(mannopyranosyl-1) phosphates of formula I, the di(manno-pyranosyl-1) phosphates of formula II and the tri(mannopyranosyl-1) phosphates of formula III′, as novel industrial products, and

a method for preparing said novel products.

### PRIOR ART

The CDG syndrome is a group of recessive autosomal diseases affecting the synthesis of glycoproteins. These diseases, which are linked to various enzymatic deficiencies, result in neurological impairments which may be associated with multivisceral impairments. Their classification is based on the level of the stage which limits glycosylation. For the CDG-1 syndrome, which is statistically the one most often encountered, the impairment, which results in an insufficient intracellular N-glycosylation, is located upstream of the transfer of the oligosaccharide on the peptide chain; on the other hand, for the CDG-II syndrome, it is located downstream of said transfer. Among the CDG-I syndrome cases, the most frequent (70% of said cases) is the CDG-Ia syndrome; it is a rare disease affecting about 500 people worldwide and which is characterized by a deficiency in phosphomannomutase (PMM) activity and mutations on the PMM2 gene (i.e. the gene expressing phosphomannomutase 2) located in 16p13, the others CDG-I and CDG-II involve only a relatively small number of cases.

The intracellular metabolism is schematically represented by Muus U. et al., Eur. J. Org. Chem., 2004; 1228-1235 as follows:

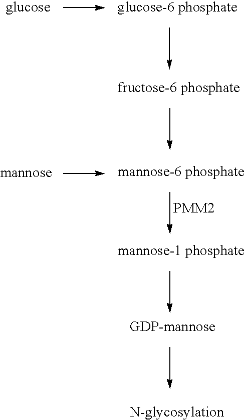

In the case of the CDG-I syndrome, and in particular that of the CDG-Ia syndrome, a deficiency at the level of the PMM2 activity causes a deficiency or insufficiency in intracellular N-glycosylation.

To overcome such a metabolism deficiency, it is appropriate to provide the cell with mannose-1 phosphate (abbreviated: Man-1 P). However, Man-1 P administered by the oral route or by injection is degraded by the enzymes of the extracellular body fluids, and nondecomposed Man-1 P, which may reach the cellular level where it is necessary, cannot penetrate the cell wall because of its high polarity due to the presence of two acidic OH groups present on the phosphorus atom, as recalled by Rutschow S. et al., Bioorg. Med. Chem., 2002; 10: 4043-4049.

Among the solutions envisaged for reducing the polarity of Man-1 P, there are known those described in:
the article by Rutschow S. et al. cited above, which corresponds to and is developed in WO 2003/104247 A (Marquardt T. et al.),
the article by Muus U. et al. cited above, and
the article by Eklund E. A. et al., Glycobiology, 2005; 15 (No. 11): 1084-1093,
   which relate to mono(mannopyranosyl-1) phosphate derivatives, in which (i) the two acid OH groups of the phosphate residue are each advantageously protected by a protecting group for the acid functional group PO—OH which can be removed, in general the same group R═R′ is used for each acid OH(PO—OH), and (ii) at least one OH group of the mannopyranosyl residue is generally protected by a removable protecting OH group, in general the 4 OH groups of said mannopyranosyl residue are protected.

The combination Rutschow S. et al./WO 2003/104247 A describes, as therapeutic agents against the CDG-Ia syndrome, extracellularly stable mono(α-D-mannopyranosyl-1) phosphates, capable of crossing the cell wall in order to provide a source at the intracellular level of Man-1 P and having a structure corresponding to formula I below, in which R₁₁, R₁₂, R₁₃ and R₁₄, which are identical or different, each represent an alkylcarbonyl, arylcarbonyl, alkyloxycarbonyl or aryloxycarbonyl group, it being additionally possible for R₁₁, R₁₂ and R₁₃ to represent H, on the one hand, and R and R′, which are identical or different, each represent an OH group or an oxymethyleneoxycarbonylalkyl [i.e. O—CH₂—O—CO-Alk] group, on the other hand, the alkyl groups having a linear or branched C₁-C₂₀ hydrocarbon chain and the aryl groups being optionally substituted aromatic hydrocarbon residues.

It happens to be the case that the compounds of said combination Rutschow S. et al./WO 2003/104247 A, (i) are relatively stable in extracellular body fluids, (ii) are capable of at least partially crossing the cell wall, with the exception of the excessively polar compounds R═R′═OH, but (iii) are cytotoxic in the sense that under the action of intracellular esterases, the abovementioned group R=oxymethyleneoxycarbonylalkyl provides formaldehyde (see in this regard scheme 1 of page 4045 of the article by Rutschow S. et al.). Thus, the higher the capacity of these products to cross the cell wall, the higher their intracellular toxicity. Such is the case for the following products of said combination, namely:
CP 1: di(pivaloyloxymethyl) (2,3,4,6-tetra-O-acetyl)-α-D-mannopyranosyl-1] phosphate,
CP2: di (pivaloyloxymethyl) (2,3,4,6-tetra-O-butyryl-α-D-mannopyranosyl-1) phosphate (compound 11 of said combination),
CP3: di(pivaloyloxymethyl) (2,3,4,6-tetra-O-pivaloyl-α-D-manno-pyranosyl-1) phosphate (compound 13 of said combination), and
CP4: di(pivaloyloxymethyl) [2,3,4,6-tetra-O-(isopropylcarbonyl)-α-D-mannopyranosyl-1] phosphate (compound 15 of said combination).

The article by Eklund E. A. et al. describes similar compounds useful against the CDG-Ia syndrome, namely:
CP5: di(acetyloxymethyl) (2,3,4,6-tetra-O-acetyl)-α-D-mannopyranosyl-1) phosphate [compound 5 (with the reference C-I) of said article], and
CP6: di(acetyloxymethyl) (2,3,4,6-tetra-O-ethyloxycarbonyl)-α-D-manno-pyranosyl-1) phosphate [compound 10 (with the reference C-II) of said article].

The article by Muus U. et al. provides another technical solution using a cyclic ester of phosphoric acid having the cyclosaligenyl-mannopyranosyl-1 phosphate structure:

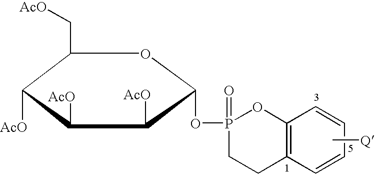

in which Q′ is H, 5-Cl, 3-Me or 3,5-diMe.

Finally, from the synthesis point of view, there are known

from the article by Colowick S. P., J. Biol. Chem., 1938: 124; 557-558, the preparation of tri[(2,3,4,6-tetra-O-acetyl)-α-D-mannopyranosyl-1] phosphate by the reaction of 1-bromo(2,3,4,6-tetra-O-acetyl)-α-D-mannopyranose with Ag₃PO₄, this article not describing the use of this product as a medicament; and

from the article by Eklund E. A. et al., the production of a mono(mannopyranosyl-1) phosphate derivative by the reaction of (2,3,4,6-tetra-O-acetyl)-α-D-mannopyranose, in the presence of Ag₂CO₃, with dibenzyl phosphate [HOP(═O)(OCH₂C₆H₅)₂].

### AIM OF THE INVENTION

According to the invention, it is proposed to provide Man-1 P derivatives which are (i) essentially stable in extracellular body fluids, (ii) capable of substantially crossing the cell wall, and (iii) intracellularly less toxic or less cytotoxic than the best prior art products which are represented by the abovementioned compounds CP1 to CP6.

Where appropriate, the reduction which is sought in the intracellular toxicity due in particular to the degradation products may result from a compromise between the capacity for penetration of said derivatives through the cell wall and the intracellular toxicity of their enzymatic degradation products.

It is also proposed to use, as novel medicaments, such Man-1 P derivatives which, as prodrugs of Man-1 P, will each play a role as an intracellular source of Man-1 P, in order to produce, by intracellular enzymatic degradation, the Man-1 P necessary in the CDG-I syndrome, and more particularly in the CDG-Ia syndrome, in order to restore the required intracellular N-glycosylation.

It is finally proposed to provide a method for preparing said Man-1 P derivatives, whether they have the structure mono(mannopyranosyl-1) phosphate, di(mannopyranosyl-1) phosphate or tri(mannopyranosyl-1) phosphate.

### OBJECT OF THE INVENTION

According to one aspect of the invention, a composition is recommended for use as a medicament, said composition containing, in combination with a physiologically acceptable excipient, an active substance chosen from the combination consisting of:

(α) the mono(α-D-mannopyranosyl-1) phosphates of formula I:

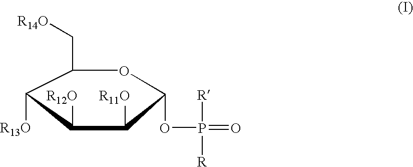

in which
R₁₁, R₁₂, R₁₃ and R₁₄, which are identical or different, each represent the hydrogen atom or an OH-protective group,
R and R′, which are identical or different, each represent
   a C₆-C₁₀ aryloxy group (in particular phenoxy, 1-naphthyloxy or 2-naphthyloxy) capable of being substituted with one or more C₁-C₅ alkyl, C₁-C₅ alkoxy, halo, CF₃ and/or nitro groups,
   an arylalkyleneoxy group (such as in particular OCH₂CH₂C₆H₅, OCH₂C₆H₅, 1-naphthylmethyloxy or 2-naphthylmethyloxy), where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
   a group having a structure:
      —O—CH(CH₃)—O—CO-alkyl, or
      —O—CH(CH₃)—O—CO—O-alkyl
   where the alkyl residue is C₁-C₅,
   a group —O—CH₂—CH(OH)—CH₂OH, where the OH groups may be protected,
   an OB residue, where B is an ethylenically unsaturated aliphatic C₂-C₂₁ residue containing a linear or branched hydrocarbon chain, or a C₅-C₂₁ cycloaliphatic residue, or
   an amino acid group having the structure VIIa:

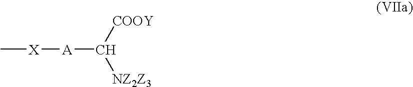

where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group,
Z₂ and Z₃, which are identical or different, each represent H, a C₁-C₅ alkyl group, or an N-protective group, or
an amino acid group having the structure VIIb:

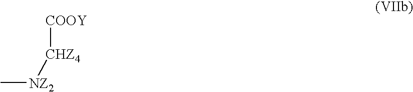

where
Y represents H or a C₂-C₅ alkyl group,
Z₄ is an alkylene, phenylene or phenylalkylene group, (where each alkylene group is C₁-C₅), or a side chain of natural amino acids, protected or not protected by a protective group,
Z₂ represents H, a C₁-C₅ alkyl group, or an N-protective group;

(β) the di(α-D-mannopyranosyl-1) phosphates of formula II:

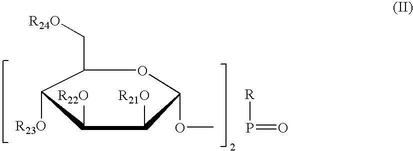

in which
R₂₁, R₂₂, R₂₃ and R₂₄, which are identical or different, each represent the hydrogen atom or an OH-protective group, and
R represents
   an OH group,
   a C₁-C₂₀ (preferably C₁-C₅) alkoxy group,
   a C₆-C₁₀ aryloxy group capable of being substituted with one or more C₁-C₅ alkyl, C₁-C₅ alkoxy, halo, CF₃ and/or nitro groups,
   an arylalkyleneoxy (in particular benzyloxy, phenylethyloxy, 1-naphthylmethyloxy or 2-naphthylmethyloxy) group, where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
   a group having the structure:
      —O—CH(Q)-O—CO-alkyl, or
      —O—CH(Q)-O—CO—O-alkyl
   where Q is H or CH₃, and the alkyl residue is C₁-C₅,
   a group —O—CH₂—CH(OH)—CH₂OH, where the OH groups may be protected,
   an OB residue, where B is an ethylenically unsaturated aliphatic C₂-C₂₁ residue containing a linear or branched hydrocarbon chain, or a cycloaliphatic C₅-C₂₁ residue, or
   an amino acid group having the structure VIIa:

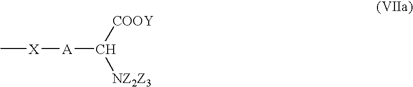

where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb:

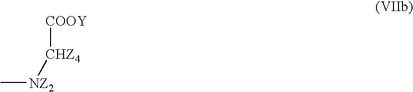

where
Y represents H or a C₂-C₅ alkyl group,
Z₄ is an alkylene, phenylene or phenylalkylene group, (where each alkylene group is C₁-C₅), or a side chain of the natural amino acids, protected or not protected with a protecting group,
Z₂ represents H, a C₁-C₅ alkyl group, or an N-protective group;

(γ) the tri(α-D-mannopyranosyl-1) phosphates of formula III:

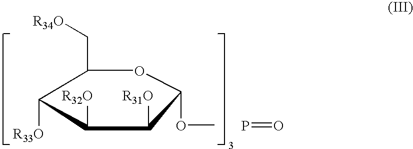

in which
R₃₁, R₃₂, R₃₃ and R₃₄, which are identical or different, each represent a hydrogen atom or an OH-protective group; and

(δ) mixtures thereof.

In this composition, said active ingredient is present in a therapeutically effective quantity and acts as an intracellular source of Man-1 P. By virtue of the R groups provided and the molecular structures envisaged, whether they are mono, di or a fortiori tri(mannopyranosyl-1) phosphate, their intracellular toxicity or cytotoxicity is very low because under the action of intracellular esterases, they do not lead to the formation of formaldehyde which is very toxic, but to the formation of other molecules which are relatively less toxic. Overall, the composition according to the invention makes it possible to obtain Man-1 P derivatives which are stable in extracellular body fluids and which can cross the cell wall, but which are especially intracellularly less toxic or cytotoxic than the best prior art products which are represented by the abovementioned compounds CP1 to CP6.

According to another aspect of the invention, the use of a (mannosyl-1) phosphate derivative is recommended, said use being characterized in that use is made of a substance acting as an intracellular source of Man-1 P, which is chosen from the combination consisting of the compounds

(α) mono(α-D-mannopyranosyl-1) phosphates of formula I,

(β) di(α-D-mannopyranosyl-1) phosphates of formula II,

(γ) tri(α-D-mannopyranosyl-1) phosphates of formula III, and

(δ) mixtures thereof,

for the preparation of a medicament intended for therapeutic use against the CDG-I syndrome, and in particular against the CDG-Ia syndrome.

According to yet another aspect of the invention, there is provided as novel industrial product a (mannosyl-1) phosphate derivative, which can be used against the CDG-I syndrome and in particular against the CDG-Ia syndrome, characterized in that it is chosen from the combination consisting of:

(α) mono(α-D-mannopyranosyl-1) phosphates of formula I:

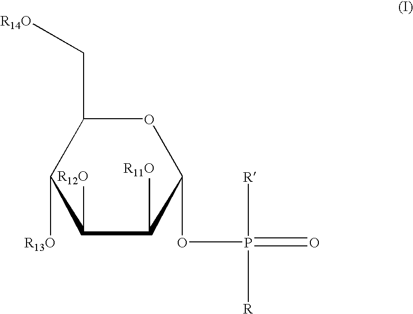

in which
R₁₁, R₁₂, R₁₃ and R₁₄, which are identical or different, each represent the hydrogen atom or an OH-protective group,
R and R′, which are identical or different, each represent
   a C₆-C₁₀ aryloxy group (in particular phenoxy, 1-naphthyloxy or 2-naphthyloxy) capable of being substituted with one or more C₁-C₅ alkyl, C₁-C₅ alkoxy, halo, CF₃ and/or nitro groups,
an arylalkyleneoxy group (in particular OCH₂CH₂C₆H₅, OCH₂C₆H₅, 1-naphthylmethyloxy or 2-naphthylmethyloxy), where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
a group having a structure:
   —O—CH(CH₃)—O—CO-alkyl, or
   —O—CH(CH₃)—O—CO—O-alkyl
where the alkyl residue is C₁-C₅,
a group —O—CH₂—CH(OH)—CH₂OH, where the OH groups may be protected,
an OB residue, where B is an ethylenically unsaturated aliphatic C₂-C₂₁ residue containing a linear or branched hydrocarbon chain, or a C₅-C₂₁ cycloaliphatic residue, or
an amino acid group having the structure VIIa:

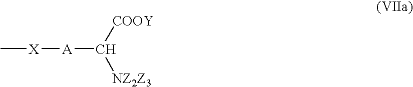

where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb:

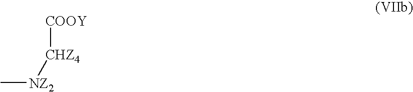

where
Y represents H or a C₂-C₅ alkyl group,
Z₄ is an alkylene, phenylene or phenylalkylene group, (where each alkylene group is C₁-C₅), or a side chain of natural amino acids, protected or not protected by a protective-group,
Z₂ represents H, a C₁-C₅ alkyl group, or an N-protective group;

(β) the di(α-D-mannopyranosyl-1) phosphates of formula II:

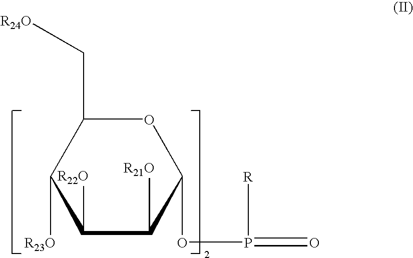

in which
R₂₁, R₂₂, R₂₃ and R₂₄, which are identical or different, each represent the hydrogen atom or an OH-protective group, and
R represents
   an OH group,
   a C₁-C₂₀ (preferably C₁-C₅) alkoxy group,
   a C₆-C₁₀ aryloxy group capable of being substituted with one or more C₁-C₅ alkyl, C₁-C₅ alkoxy, halo, CF₃ and/or nitro groups,
   an arylalkyleneoxy (in particular benzyloxy, phenylethyloxy, 1-naphthylmethyloxy or 2-naphthylmethyloxy) group, where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
   a group having the structure:
      —O—CH(O)—O—CO-alkyl, or
      —O—CH(O)—O—CO—O-alkyl
   where Q is H or CH₃, and the alkyl residue is C₁-C₅,
   a group —O—CH₂—CH(OH)—CH₂OH, where the OH groups may be protected,
   an OB residue, where B is an ethylenically unsaturated aliphatic C₂-C₂₁ residue containing a linear or branched hydrocarbon chain, or a cycloaliphatic C₅-C₂, residue, or
   an amino acid group having the structure VIIa:

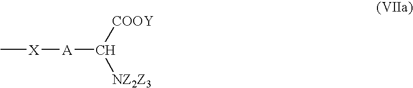

where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb:

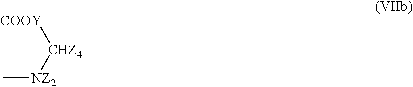

where
Y represents H or a C₂-C₅ alkyl group,
Z₄ is an alkylene, phenylene or phenylalkylene group, (where each alkylene group is C₁-C₅), or a side chain of the natural amino acids, protected or not protected with a protecting group,
Z₂ represents H, a C₁-C₅ alkyl group, or an N-protective group;

(γ) the tri(α-D-mannopyranosyl-1) phosphates of formula III′:

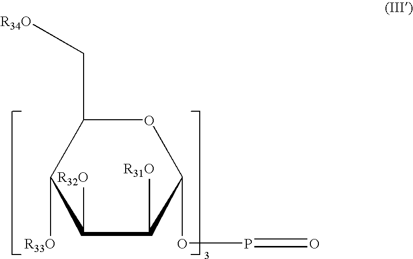

in which
R₃₁, R₃₂, R₃₃ and R₃₄, which are identical or different, each represent an OH-protective group having at least three carbon atoms; and

(δ) mixtures thereof.

Finally, according to another aspect of the invention, there is provided a method for preparing a compound of formula I, II or III′, said method being characterized in that it comprises

(a) the reaction of a 1-bromomannopyranose of formula (IVa):

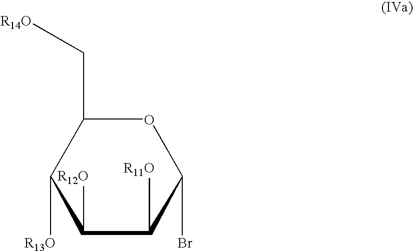

where R₁₁, R₁₂, R₁₃ and R₁₄ are defined as indicated above, with a monosilver phosphate of formula (Va):

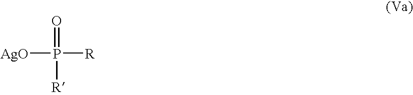

where R and R′ are defined as indicated above,

in order to obtain a mono(α-D-mannopyranosyl-1) phosphate compound of formula I;
(b) the reaction of a 1-bromomannopyranose of formula (IVb):

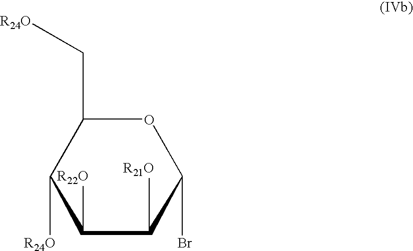

where R₂₁, R₂₂, R₂₃ and R₂₄ are defined as indicated above, with a disilver phosphate of formula (Vb):

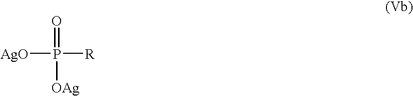

where R is defined as indicated above,
in order to obtain a di(α-D-mannopyranosyl-1) phosphate compound of formula II; or
(c) the reaction of a 1-bromomannopyranose of formula (IVc):

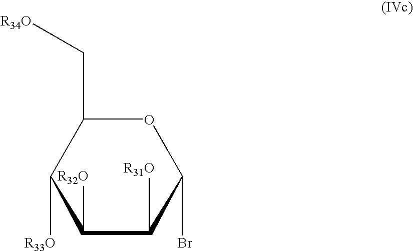

where R₃₁, R₃₂, R₃₃ and R₃₄, which are identical or different, each represent an OH-protective group which is a C₃-C₆ acyl group, with a trisilver phosphate of formula (Vc):

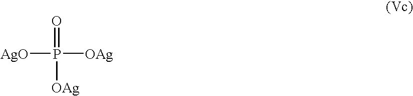

in order to obtain a tri(α-D-mannopyranosyl-1) phosphate compound of formula III′.

in this method, the optional customary operations of protecting, deprotecting and then reprotecting the hydroxyl groups of the mannopyranosyl-1 residue and/or of the acid OH groups of PO—OH were omitted for convenience.

As a variant, the silver phosphate of formula Va, Vb or, respectively, Vc may be replaced by a phosphate of formula VIa, VIb or, respectively, VIC:

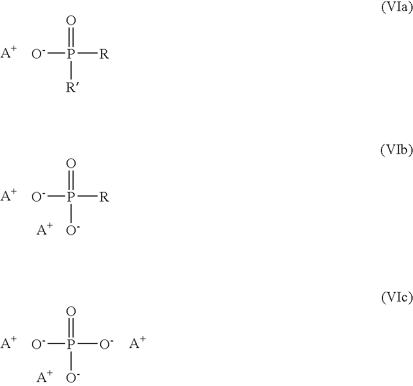

in which R and R′ are defined as indicated above, and A is H or R″₄N, R″ being H or an N-alkyl, cycloalkyl or aromatic group.

### DETAILED DESCRIPTION OF THE INVENTION

The expression halo group is understood to mean here a halogen atom such as F, Cl, Br or I. From the synthesis point of view, the preferred halogens are Cl and especially Br. From the point of view of the pharmacological properties, the preferred halo groups on the aromatic groups are F and Cl. Moreover, the CF₃ group is also a substituent which is of some interest in terms of the pharmacological properties.

The OH-protective groups, which act according to the invention in order to protect at least one hydroxyl group of the α-D-mannopyranosyl residue, are groups which are customarily used in the field of chemical syntheses in particular (i) in that of sugars and (ii) in that of peptides containing hydroxylated side groups. These protecting groups can in general be removed in order to restore the hydroxyl group(s) involved in the protection. Among the OH-protective groups which are suitable here, there may be mentioned in particular the acyl, in particular C₂-C₂₀ acyl, groups which are aliphatic, aromatic or arylaliphatic, in particular of the type:
—CO-alkyl (where the alkyl group is C₁-C₁₉ and preferably C₁-C₅),
—CO-aryl (where the aryl group is preferably C₆-C₁₀ and may be substituted with one or more C₁-C₅ alkyl, C₁-C₅ alkoxy, halo, CF₃ and/or nitro groups) and
—CO-alkylenearyl (where the alkylene group is advantageously C₁-C₅, and the aryl group is C₆-C₁₀ and is capable of being substituted with one or more C₁-C₅ alkyl, C₁-C₅ alkoxy, halo, CF₃ and/or nitro groups).

Advantageously, in the formulae I, II and III, the OH-protective group for the OH functional groups at the 2-, 3-, 4- and 6-positions of the mannosyl ring is mainly an aliphatic C₂-C₆ acyl group (in particular COCH₃, COCH₂CH₃, COCH₂CH₂CH₃, COCH(CH₃)₂, CO(CH₂)₃CH₃, COC(CH₃)₃, COCH(CH₃)CH₂CH₃ or COCH₂CH(CH₃)₂].

In formula III′, the OH-protective group for the OH functional groups at the 2-, 3-, 4- and 6-positions of the mannosyl ring is mainly (i) an aliphatic C₃-C₆ acyl group, in particular COCH₂CH₃, COCH₂CH₂CH₃, COCH(CH₃)₂, CO(CH₂)₃CH₃, COC(CH₃)₃, COCH(CH₃)CH₂CH₃ or COCH₂CH(CH₃)₂.

The protective groups for the OH group(s) of the acid functional group PO—OH, which are involved according to the invention, are also conventional in the field of organic chemistry. The relevant protection, which is optionally temporary, is mainly obtained by esterification of the acid functional group PO—OH by means of a compound having a hydroxyl group of the alcohol (or derivative) or phenol (or derivative) type. Examples of protecting groups for each acid functional group PO—OH (i.e. when R and/or R′═OH) are given later. Finally, the N-protective groups, which are involved here in the structure VII, are well known in peptide chemistry.

The R group of formulae I and II (and this same applies for the R′ group of formula I) represents in general:
an OH group (in particular for the synthesis of other mannopyranosyl-1 phosphates),
an OT group, where T is a protective residue for the acid functional group PO—OH, or
an amino residue (in particular when R is a residue of structure VII, in which the group X is —NZ₁-).

Accordingly, the R group according to the invention is:
(a) a C₁-C₂₀ alkoxy group (i.e. T=C₁-C₂₀ alkyl), when this is a compound of formula II, preferably as C₁-C₅,
(b) a C₆-C₁₀ aryloxy group (i.e. T=C₆-C₁₀ aryl) which is capable of being substituted with one or more C₁-C₅ alkyl, C₁-C₅ alkoxy, halo, CF₃ and/or nitro groups, in particular a phenoxy, 4-methoxyphenoxy, 3,4-dimethoxyphenoxy, 4-nitrophenoxy, 3-chlorophenoxy, 4-chlorophenoxy, 3,5-dimethylphenoxy, 3-trifluoromethylphenoxy, 1-naphthyloxy or 2-naphthyloxy group,
(c) a (C₁-C₅)aryl(C₆-C₁₀)alkyleneoxy group, in particular a benzyloxy, phenethyloxy, 1-naphthylmethyloxy or 2-naphthylmethyloxy group, where the aryl residue may be substituted with one or more C₁-C₅ alkyl, C₁-C₅ alkoxy, halo, CF₃ and/or nitro groups,
(d) a group

—O—CH(O)—O—CO—(C₁-C₅)alkyl
where Q is CH₃ in formula I, and H or CH₃ in formula II, or

—O—CH(O)—O—CO—O—(C₁-C₅)alkyl,
where Q is H or CH₃,
(e) a group —O—CH₂—CH(OH)—CH₂OH, where the OH groups may be protected,
(f) a group OB, where B is an ethylenically unsaturated (which may contain one or more double bonds C═C), aliphatic C₂-C₂₁ residue containing a linear or branched hydrocarbon chain, or a cycloaliphatic C₅-C₂₁ residue, in particular a group —O—CH₂—CH═C(CH₃)₂ or a terpeneoxy group in which the terpene portion is cyclic or acyclic, among the acyclic groups R=terpeneoxy which are suitable, there may be mentioned without limitation: the farnesyloxy group having a structure (VIII):

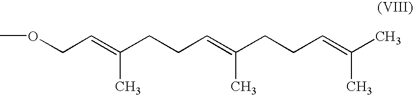

[other nomenclature: (3,7,11-trimethyl-2,6,10-dodecatriene-1-yl)oxy], and
the geranyloxy group having the structure (IX):

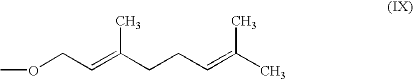

[other nomenclature: ((E)-3,7-dimethyl-2,6-octadiene-1-yl)oxy],
(g) a group having the structure VIIa:

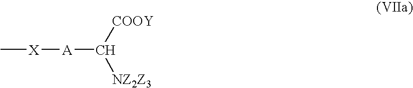

where X is —O—, —S— or —NZ₁-, and, A, Y, Z₂ and Z₃ are defined as indicated above, and
(h) an amino acid group having the structure VIIb:

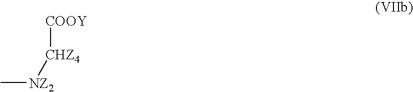

where
Y represents H or a C₂-C₅ alkyl group,
Z₄ is an alkylene, phenylene or phenylalkylene group, (where each alkylene group is C₁-C₅) or a side chain of natural amino acids, which is protected or not protected with a protecting group,
Z₂ represents H, a C₁-C₅ alkyl group, or an N-protective group.

Advantageously, said structures VIIa, VIIb may be prepared from an amino acid (advantageously a natural amino acid) containing an amine or hydroxyl side functional group allowing the attachment of the basic or hydroxylated amino acid to the phosphorus atom.

Among the amino acids which are suitable, the amino acids with a basic side chain such as lysine, on the one hand, and the amino acids with a hydroxylated side chain such as tyrosine, serine and threonine, on the other hand are recommended. It will be noted that cysteine or homocysteine, like serine or homoserine, are amino acids which are also suitable.

The preferred R (or R′) groups according to the invention are the following as regards the compounds of formula I or II:
(α) a phenoxy or 1-naphthyloxy group,
(β) a benzyloxy or 1-naphthylmethoxy group,
(γ) a group —O—CH(Q)-O—CO—O—(C₁-C₅)alkyl, where Q is H or CH₃, excluding H for the compound of formula I in order to avoid the formation of formaldehyde during the degradation of the compound,
(δ) a group —O—CH₂—CH(OH)—CH₂OH, where the OH groups may be protected,
(ε) a group εLys, pTyr; βSer or βThr, whose structures (where the NH₂ or COOH groups may be protected) are the following:
   εLys: —NH—(CH₂)₄—CH(NH₂)COOH,
   pTyr: -(p-O)—C₆H₄—CH₂—CH(NH₂)COOH,
   βSer: —O—CH₂—CH(NH₂)COOH, and
   βThr: —O—CH(CH₃)—CH(NH₂)COOH, and
(ξ) a group
   —NH—(CH₂)₃—CH(NH₂)COOH or
   —NH—(CH₂)₂—CH(NH₂)COOH,
   where the NH₂ or COOH functional groups may be protected.

As regards the compounds of formula II, there may also be mentioned another preferred group R:
(η) —O—CH(Q)-O—CO—(C₁-C₅)alkyl, where Q is H or CH₃.

As indicated above, the invention relates to in particular (a) as medicaments, the mono(α-D-mannopyranosyl-1) phosphates of formula I, the di(α-D-mannopyranosyl-1) phosphates of formula II and the tri(α-D-mannopyranosyl-1) phosphates of formula III:

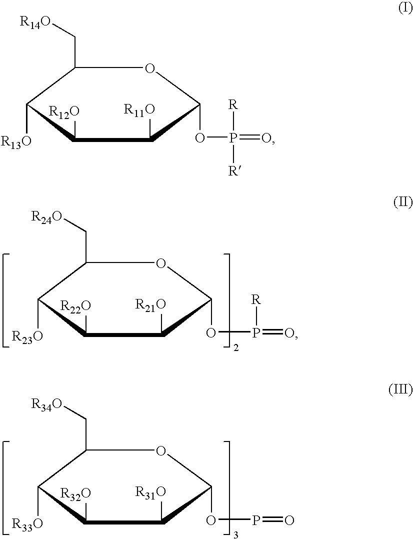

and (b) as novel industrial products, the mono(α-D-mannopyranosyl-1) phosphates of formula I, the di(α-D-mannopyranosyl-1) phosphates of formula II and the tri(α-D-mannopyranosyl-1) phosphates of formula III′ above.

The compounds of formula I where R₁₁═R₁₂═R₁₃═R₁₄═H, and R═R′═OH, those of formula II where R₂₁═R₂₂═R₂₃═R₂₄═H and R═OH, and those of formula III where R₃₁═R₃₂═R₃₃═R₃₄═H are (i) useful from the point of view of the synthesis of other compounds of the invention and (ii) advantageous from the pharmacological point of view.

However, the compounds of formulae I, II and III, where all the OH groups are protected both on the mannopyranosyl ring and on the phosphorus atom, act more effectively than the previous ones as intracellular sources of Man-1 P: after having crossed the cell wall, they are mainly deprotected by the enzymatic route in order to provide the Man-1 P required in the CDG-I syndrome and more particularly in the CDG-Ia syndrome.

Practically, for the treatment of patients suffering from the CDG-Ia syndrome-, the compounds according to the invention which are the most advantageous are (in decreasing order of interest) the following:

(1°) the di(2,3,4,6-tetra-O-acyl-α-D-mannopyranosyl-1) phosphates of formula II, in which the OH-protective group for the hydroxyl groups at the 2-, 3-, 4- and 6-positions of the mannopyranosyl ring is such that: R₂₁═R₂₂═R₂₃═R₂₄=aliphatic C₂-C₆ acyl;
(2°) the mono(2,3,4,6-tetra-O-acyl-(α-D-mannopyranosyl-1) phosphates of formula I, in which R═R′ and the OH-protective group for the hydroxyl groups at the 2-, 3-, 4- and 6-positions of the mannopyranosyl ring is such that: R₁₁═R₁₂═R₁₃═R₁₄=aliphatic C₂-C₆ acyl; and then
(3°) the tri(2,3,4,6-tetra-O-acyl-α-D-mannopyranosyl-1) phosphates of formula III, in which the OH-protective group for the hydroxyl groups at the 2-, 3-, 4- and 6-positions of the mannopyranosyl ring is such that: R₃₁═R₃₂═R₃₃═R₃₄=aliphatic C₂-C₆ acyl.

In tables I, II and III, which follow, a number of typical (α-D-mannopyranosyl-1) phosphate derivatives according to the invention have been presented. For the sake of convenience, the groups R₁₁ to R₁₄, R₂₁ to R₂₄ and R₃₁ to R₃₄, are represented therein by the symbol R₁, on the one hand, and the formulae I, II and, respectively, III are designated by Ia, IIa and, respectively, IIIa, on the other hand.

In these tables I, II and III, the abbreviations used are the following:
Ac acetyl,
Bu butyl [CH₂CH₂CH₂CH₃],
iBu isobutyl [CH₂CH(CH₃)₂],
sBu sec-butyl [CH(CH₃)CH₂CH₃],
tBu tert-butyl [C(CH₃)₃],
Far farnesyl [see above],
Ger geranyl [see above],
εLys ε-lysyl residue [NH—(CH₂)₄—CH(NH₂)COOH] in which the NH₂ and COOH functional groups may be protected,
Me methyl
1 Napht 1-naphthyl
Pr propyl [CH₂CH₂CH₃],
iPr isopropyl [CH(CH₃)₂],
βSer β-serinyl residue [O—CH₂—CH(NH₂)COOH] in which the NH₂ and COOH functional groups may be protected,
βThr β-threonyl residue [O—CH(CH₃)—CH(NH₂)COOH] in which the NH₂ and COOH functional groups may be protected,
pTyr p-tyrosyl residue [(p-O—C₆H₄CH₂)—CH(NH₂)COOH] in which the NH₂ and COOH functional groups may be protected,

TABLE 1

| | |
|---|---|
| | (Ia) |
| 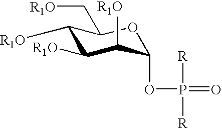 | |

| Product | R₁ | R |
|---|---|---|
| Ex. 20 | Ac | O-1Napht |
| Ex. 21 | Ac | O—C₆H₅ |
| Ex. 22 | Ac | O—CH₂C₆H₄(4-CF₃) |
| Ex. 23 | CO-tBu | O—CH₂C₆H₄(3,4-diMeO) |
| Ex. 24 | CO-sBu | O—CH(CH₃)—O—CO—CH₂CH₃ |
| Ex. 25 | CO-iPr | O—CH₂—O—CO—O-tBu |
| Ex. 26 | CO-Bu | O—CH₂—O—CO—O-iPr |
| Ex. 27 | Ac | εLys |
| Ex. 28 | Ac | pTyr |
| Ex. 29 | Ac | O—CH(CH₃)—O—CO—O-iPr |
| Ex. 30 | Ac | O-Far |
| Ex. 31 | Ac | O-Ger |
| Ex. 32 | Ac | O—CH₂—CH═C(CH₃)₂ |
| Ex. 33 | Ac | O—(CH₂)₂—CH═C(CH₃)₂ |

TABLE II

| | |
|---|---|
| | (IIa) |
| 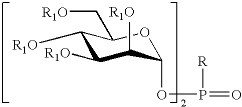 | |

| Product | R₁ | R |
|---|---|---|
| Ex. 1 | Ac | O—CH₂CH₃ |
| Ex. 2 | Ac | O—CH₂C₆H₅ |
| Ex. 3 | Ac | O—CH₂C₆H₄(4-NO₂) |
| Ex. 4 | CO-iPr | OH |
| Ex. 5 | Ac | O—C₆H₅ |
| Ex. 6 | CO-tBu | O—CH₂C₆H₄(4-OCH₃) |
| Ex. 7 | CO-tBu | O—CH₂-1Napht |
| Ex. 8 | CO-tBu | O—C₆H₅ |
| Ex. 9 | CO-tBu | O—CH₂—O—CO-tBu |
| Ex. 10 | CO-tBu | εLys |
| Ex. 11 | Ac | O—CH₂-1Napht |
| Ex. 12 | CO-iPr | O—CH₂C₆H₅ |
| Ex. 13 | CO-iPr | O—C₆H₅ |
| Ex. 14 | Ac | βThr |
| Ex. 15 | CO-Pr | βSer |
| Ex. 16 | CO-iPr | pTyr |
| Ex. 17 | Ac | εLys |
| Ex. 18 | Ac | O-Far |
| Ex. 19 | Ac | O-Ger |
| Ex. 20 | Ac | O—CH₂—CH═C(CH₃)₂ |
| Ex. 21 | Ac | O—(CH₂)₂—CH═C(CH₃)₂ |

TABLE III

| | |
|---|---|
| | (IIIa) |
| 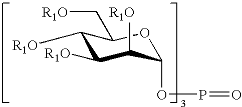 | |

| Product | R₁ |
|---|---|
| Ex. 33 | COCH₃ |
| Ex. 34 | CO—CH₂CH₃ |
| Ex. 35 | CO-Pr |
| Ex. 36 | CO-iPr |
| Ex. 37 | CO-tBu |
| Ex. 38 | CO-iBu |
| Ex. 39 | CO-Bu |

The compounds of formula I, II or III may be prepared according to a method known per se by application of conventional reaction mechanisms. The method of synthesis, which is recommended according to the invention, uses the nucleophilic substitution reactions (1), (2) or (3) which follow:

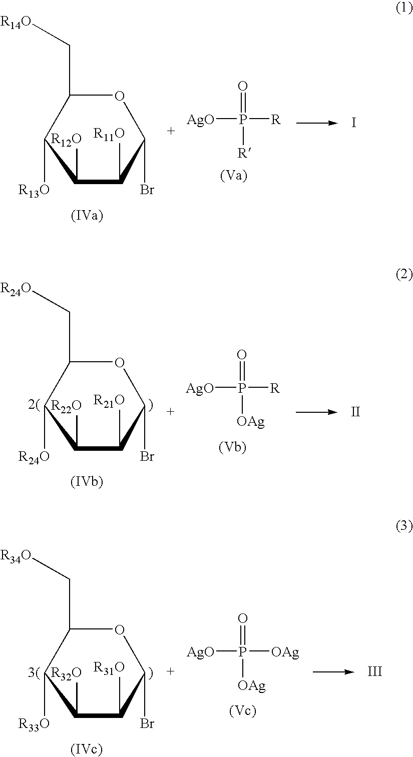

Each of these reactions (1), (2) and (3) is carried out at a temperature of 15 to 40° C., preferably at room temperature (RT=15-25° C.) in an appropriate inert solvent, in the presence of a molecular sieve. Such a solvent is advantageously toluene. The molecular sieve which is advantageously recommended is a 4 Å (i.e. 0.4 μm) molecular sieve.

Advantageously, the protection, deprotection and then reprotection operations are included in the reaction mechanisms for the method of preparation according to the invention, namely:

(1°) protection of the OH groups at the 2-, 3-, 4- and 6-positions of the mannopyranosyl residue during the synthesis of the compounds of formula IVa, IVb or IVc, upstream of the reactions (1), (2) or (3), by acylation of said OH groups;
(2°) protection of the acid group(s) R (or R′)═OH bound to the phosphorus atom in the formula Va, Vb or Vc, for example by means of a group R (or R′)=alkoxy (in particular ethyloxy), aryloxy (in particular phenoxy, 1-naphthyloxy or 2-naphthyloxy) or arylalkyloxy (in particular benzyloxy, 1-naphthylmethoxy or 2-naphthylmethoxy), upstream of the reaction (1), (2) or (3), by esterification of the acid groups PO—OH with an alcohol or a derivative;
(3°) carrying out of the reaction (1), (2) or (3);
(4°) where appropriate, deprotection of the alkoxy group R (or R′) different from OH in order to obtain the acid functional group(s) PO—OH;
(5°) reprotection of the acid group(s) R═OH, thus obtained, by esterification reaction with an alcohol or a derivative different from that of step (2°) or, respectively, by amidation reaction with an amine in order to obtain novel amides of the PO-NZ₁-A-CH(NH₂)COOH type (where Z₁ is defined as above, and the NH₂ and COOH functional groups may be protected).

In practice, it is not necessary to envisage the protection, deprotection and then reprotection of the OH groups at the 2-, 3-, 4- and 6-positions of the mannopyranosyl residue. It is enough to start with a compound of formula IVa, IVb or IVc containing the desired final acyl residue at the 2, 3, 4 and 6 positions.

In order not to complicate the modes of synthesis, it is preferable to have instead:

### (1°) R₁₁═R₁₂═R₁₃═R₁₄,

R₂₁═R₂₂═R₂₃═R₂₄, and
R₃₁═R₃₂═R₃₃═R₃₄, on the one hand; and
   (2°) R═R′, on the other hand.

Briefly, a derivative of (α-D-mannopyranosyl-1) phosphate is recommended according to the invention, which is characterized in that:
in formula I, R═R′, on the one hand, and the OH-protective group for the hydroxyl groups at the 2-, 3-, 4- and 6-positions of the mannopyranosyl ring is such that: R₁₁═R₁₂═R₁₃═R₁₄═C₂-C₆ acyl, on the other hand;
in formula II, the OH-protective group for the hydroxyl groups at the 2-, 3-, 4- and 6-positions of the mannopyranosyl ring is such that: R₂₁═R₂₂═R₂₃═R₂₄═C₂-C₆ acyl; and
in formula III, the OH-protective group for the hydroxyl groups at the 2-, 3-, 4- and 6-positions of the mannopyranosyl ring is such that: R₃₁═R₃₂═R₃₃═R₃₄═C₂-C₆ acyl.

The products of the invention may be administered to patients suffering from the CDG-Ia syndrome by the oral route which is the simplest route to use and the most appropriate in the majority of patients. According to the current state of knowledge, a daily dose delivering about 300 to 750 mg/kg of body weight of Man-1 P appears to be indicated, given the mannose doses used in the treatment per os of the CDG-Ib syndrome whose enzymatic deficiency is just upstream in the metabolic sequence leading to mannose-6 phosphate, which is the substrate for PPM2.

As indicated above, the (α-D-mannopyranosyl-1) phosphate derivatives, in which R (or R′)═OH, can be used as intermediates for the synthesis of the compounds of formula I or II where R is different from OH.

Other advantages and characteristics of the invention will be understood more clearly on reading the description which follows (i) of examples of preparation and (ii) of results of pharmacological trials. Of course, all these elements are not at all limiting but are provided by way of illustration.

### Preparation I

### Silver Monoethyl Phosphate

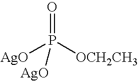

Phosphorous acid (5 g; 61 mmol) is solubilized in a solution of ethanol (53 mL) and triethylamine (30 mL; 6.75 mmol). Iodine (23.2 g; 91.4 mol) is then added in portions to the solution cooled to 5° C. After stirring for 30 minutes, the mixture is poured into acetone (400 mL) at 0° C. and an excess of cyclohexylamine (20 mL) is added. The precipitate formed is filtered, washed with acetone and recrystallized from hot ethanol. The yield of dicyclohexylammonium ethyl phosphate is 80%.

A solution of dicyclohexylammonium ethyl phosphate in distilled water is exchanged on a Dowex 50W×8-100 column (5×3.5 cm) in the Na+ form. The resin is washed with 5 volumes of pure water relative to the resin. After concentrating the eluates, the product in the sodium salt form (1.684 mg; 1 mmol) is taken up in water (2 mL) and a solution of AgNO₃ (378.8 mg; 2.23 mmol) in water (2 mL) is added. The mixture is stirred in the dark, at room temperature. The precipitate formed is then filtered, successively rinsed with water at 0° C., ethanol and ether, and then dried. The silver monoethyl phosphate thus obtained is stored at −20° C.

### Preparation II

### Ex. 1

### Ethyl di[2,3,4,6-tetra-O-acetyl-α-D-mannopyranosyl-1] phosphate

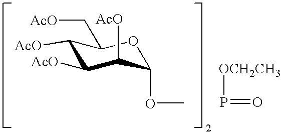

The silver monoethyl phosphate (333 mg; 0.9 mmol), obtained according to the method of Preparation I, is stirred, in suspension in anhydrous toluene (3 ml) with an activated 4 Å molecular sieve, for 30 minutes at a temperature of 15-20° C. A solution of 1-bromo(2,3,4,6-tetra-O-acetyl)-α-D-mannopyranose (732.3 mg; 1.78 mmol) in toluene (2 mL) is added and the mixture is stirred at room temperature for 6 h. After filtration on celite and evaporation of the toluene, the mixture is purified by chromatography on a silica column (eluent: cyclohexane/ethyl acetate: 5/5 v/v with 3‰ of triethylamine). The expected product is obtained pure with a yield of 65% (455 mg; 0.59 mmol).

[α]_{D}²⁰=+38 (c 1.0; CH₂Cl₂)

¹H NMR (CDCl₃, 250 MHz): δ 5.72 (d, 2H, J_{1,2}=5.5 Hz, H-1), 5.32-5.39 (m, 6H, H-2, H-3, H-4), 4.12-4.42 (m, 8H, H-5, 2H-6, CH₂—CH₃), 2.02; 2.08; 2.12; 2.19 (s, 24H, 4 acetyls), 1.43 (t, 3H, J=7.25 Hz, CH₂—CH₃)

¹³C-NMR (CDCl₃, 63 MHz): δ 170.6; 169.9; 169.8; 169.6 (CO acetates), 95.6; 95.5 (2 d, J_{C1-P}=6.3 Hz, C-1^{A}, C-1^{B}), 70.8; 70.6 (C-5^{A}, C-5^{B}), 68.9; 68.7 (2 d, J_{C2-P}=3 Hz, C-2^{A}, C-2^{B}), 68.2 (C-4^{A}, C-4^{B}), 65.5 (d, J_{C-P}=6.3 Hz, CH₂CH₃), 65.4; 65.2 (C-3^{A}, C-3^{B}), 62.0; 61.8 (C-6^{A}, C-6^{B}), 20.7 (CH₃ acetates), 16.2 (d, J_{C-P}=6.2 Hz, CH₃CH₂)

³¹P-NMR (CDCl₃, 250 MHz): δ −15 ppm

Electrospray HRMS (positive mode): calculated for C₃₀H₄₇O₂₂NP [M+NH4]⁺: 804.2327; found: 804.2329.

### Preparation III

### Ex. 12

### Benzyl di(2,3,4,6-tetra-O-isobutyryl-α-D-mannopyranosyl-1) phosphate

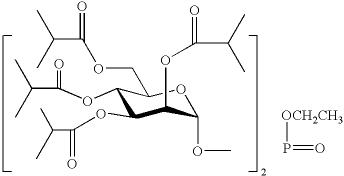

Disilver benzyl phosphate (148.08 mg; 0.368 mmol) in suspension in anhydrous toluene (3 mL) with an activated 4 Å molecular sieve (0.5 g) is stirred for 30 min at room temperature. Next, a solution of 1-bromo-(2,3,4,6-tetra-O-isobutyryl)-α-D-mannopyranose (350 mg; 0.67 mmol) is added under argon and the mixture is stirred at room temperature overnight. After filtration and evaporation of the solvents, the residue taken up in CH₂Cl₂ is purified on a Sephadex LH-20 column and eluted with a CH₂Cl₂/MeOH mixture: 7/3 v/v. The expected product is isolated with a yield of 80% (280 mg; 0.26 mmol).

[α]_{D}²⁰=+44 (c 1.0, CH₂Cl₂)

¹H NMR (CDCl₃, 250 MHz): δ 5.71 (d, 1H, J_{H1a-P}=7.5 Hz, H-1^{A}), 5.69 (dd, 1H, J_{H1B-P}=5.6 Hz, J_{H1B-2}=1.5 Hz, H-1^{B}), 5.52; 5.47 (2 dd, 2H, J_{H4-5}=10 Hz, J_{H4-3}=10 Hz, H-4), 5.42-5.34 (m, 2H, H-3), 5.29 (t, 2H, J_{H2-3}=2.5 Hz, H-2), 5.19 (d, 2H, J=8.8 Hz, CH₂ benzyl), 4.40 (dd, 1H, J_{H6a-5a}=2.5 Hz, J_{H6a-6b}=12.5 Hz, H-6^{A}), 4.27 (d, 1H, J_{H5b-4}=10 Hz, H-5^{B}), 4.15-4.00 (m, 3H, H-5^{A}, H-6^{B}, H-6′^{A}), 3.90 (d, 1H, J_{H6′a-6′b}=10 Hz, H-6′^{B}), 2.64-2.36 (m, 8H, CH(CH₃)₂), 1.26-1.00 (m, 48H, CH(CH₃)₂).

¹³C-NMR (CDCl₃, 63 MHz): δ 176.7; 176.6; 175.8; 175.6 (CO isobutyrates), 135.1 (d, J_{C-P}=6.8 Hz, aromatic C_{quat}), 129.4; 129.2; 128.6 (aromatic CH), 96.0; 96.3 (C-1^{A}, C-1^{B}), 71.4; 71.2 (C-5^{A}, C-5^{B}), 70.9 (d, J_{C-P}=6.3 Hz, CH₂Ph), 68.7 (C-2, C-3), 64.5 (C-4), 61.2 (C-6), 34.2 (CH(CH₃)₂), 19.3; 19.1 (CH(CH₃)₂)

³¹P-NMR (CDCl₃, 250 MHz): δ −15.5 ppm

Electrospray HRMS (positive mode): calculated for C₅₁H₇₇O₂₂PNa [M+Na]⁺: 1095.4542; found: 1095.4521

### Preparation IV

### Ex. 4

### Di(2,3,4,6-tetra-O-isobutyryl-α-D-mannopyranosyl-1) hydrogen phosphate

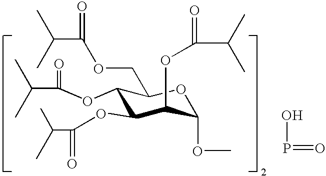

The benzyl di (2,3,4,6-tetra-O-isobutyryl-α-mannopyranosyl-1) phosphate (48 mg; 44.8 μmol), obtained according to the method of Preparation III, is stirred in methanol (2 mL) in the presence of 10% Pd/C (20 mg) under an H₂ atmosphere for 5 h. The reaction mixture is then filtered and the solvents evaporated. The expected debenzylated product is obtained with a yield of 93% (41 mg; 41.7 μmol)

[α]_{D}²⁰=+9 (c 1.0; CH₂Cl₂).

¹H NMR (CDCl₃, 250 MHz): δ 5.58 (d, 2H, H-1, J_{H1-P}=5 Hz), 5.20-5.50 (m, 6H, H-2, H-3, H-4), 4.32 (dl, 4H, J_{H6-6′}=10 Hz, H-6, H-6′), 4.20 (m, 2H, H-5), 2.69-2.41 (m, 8H, CH(CH₃)₂), 1.27-1.00 (m, 48H, CH(CH₃)₂)

¹³C-NMR (CDCl₃, 63 MHz): δ 175.7; 176.4; 177.0 (CO isobutyrates), 94.4 (C-1), 70.0-69.0 (C-2, C-3, C-5), 66.0 (C-4), 62.0 (C-6), 34.2 (CH(CH₃)₂), 18.8; 19.2; 19.4 (CH(CH₃)₂)

³¹P-NMR (CDCl₃, 250 MHz): δ −20 ppm

Electrospray HRMS (positive mode): calculated for C₄₄H₇₀O₂₂PNa₂ [M-H+2Na]⁺: 1027.3892; found: 1027.3905

### Pharmacological Trials

The compounds of formula I, II and III were tested as prodrugs (i.e. as intracellular sources of Man-1 P), on the one hand, in order to evaluate their toxicity and, on the other hand, in order to assess their capacity to inhibit the incorporation of 2-[³H]mannose into cellular glycoconjugates. Indeed, while they can generate Man-1 P in cells, it will be in competition with 2-[³H]mannose-1 phosphate for entering the pathway for biosynthesis of glycoproteins (see Eklund, E. A., et al. cited above).

The first results obtained are presented in Table IV which follows. The products were tested (5 trials per product and per dose) on lymphoblasts of CDG-Ia diseases and their activities (“radioactivity”, i.e. inhibition, by competition, of the binding of 2-[³H]mannose-1 phosphate; and cell “toxicity”) were assessed as a percentage relative to the controls (10 trials).

These results show that the best prior art products CP1, CP3 and CP6 (at the dose of 500 μM) inhibit by at least 90% the incorporation of 2[³H]mannose into the glycoproteins. Nevertheless, this effect is accompanied by at least a doubling of cell mortality. All these effects were observed on lymphoblasts derived from healthy subjects and CDG-Ia patients. On the other hand, for the products of examples 5, 11, 12 and 13 (used at the dose of 500 μM), the first results are very favorable. Indeed, although Ex. 5, Ex. 11, Ex. 12 and Ex. 13 are less effective than CP1, CP3 and CP6 in inhibiting the incorporation of radioactive mannose into glycoproteins, they are however a lot less toxic than them. The product of example 4, which is an acid product of formula II (where R═OH), is less toxic and therapeutically more advantageous than CP1, CP3 and CP6; nevertheless, it appears to be therapeutically less effective than Ex. 5, Ex. 11, Ex. 12 and Ex. 13.

TABLE IV

| Products | Radioactivity | Toxicity |
|---|---|---|
| CP1 | 92% | 210% |
| CP3 | 90% | 205% |
| CP6 | 91% | 201% |
| Ex. 4* | 100% | 100% |
| Ex. 5 | 80% | 120% |
| Ex. 11 | 40% | 115% |
| Ex. 12 | 25% | 112% |
| Ex. 13 | 21% | 110% |

Remark
*acid compound (R = OH)

1. A composition for use as a medicament, wherein it contains, in combination with a physiologically acceptable excipient, an active substance chosen from the combination consisting of:
(a) the mono(α-D-mannopyranosyl-1) phosphates of formula I: 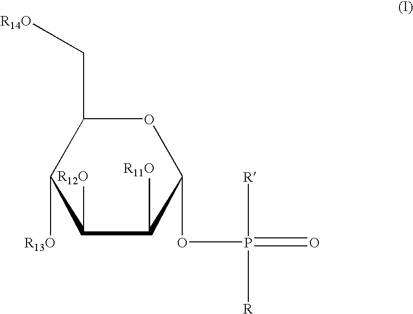
in which
R₁₁, R₁₂, R₁₃ and R₁₄, which are identical or different, each represent the hydrogen atom or an OH-protective group,
R and R′, which are identical or different, each represent
a C₆-C₁₀ aryloxy group capable of being substituted with one or more C₁-C₅ alkyl, C₁-C₅ alkoxy, halo, CF₃ and/or nitro groups,
an arylalkyleneoxy group, where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
a group having a structure:
—O—CH(CH₃)—O—CO-alkyl, or
—O—CH(CH₃)—O—CO—O-alkyl
where the alkyl residue is C₁-C₅,
a group —O—CH₂—CH(OH)—CH₂OH, where the OH groups may be protected,
an OB residue, where B is an ethylenically unsaturated aliphatic C₂-C₂₁ residue containing a linear or branched hydrocarbon chain, or a C₅-C₂₁ cycloaliphatic residue, or
an amino acid group having the structure VIIa: 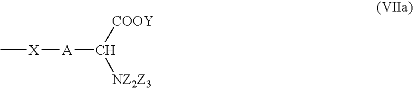
where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group,
Z₂ and Z₃, which are identical or different, each represent H, a C₁-C₅ alkyl group, or an N-protective group;
an amino acid group having the structure VIIb: 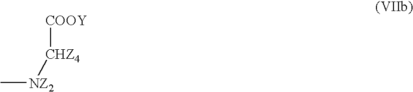
where
Y represents H or a C₂-C₅ alkyl group,
Z₄ is an alkylene, phenylene or phenylalkylene group, (where each alkylene group is C₁-C₅),
Z₂ represents H, a C₁-C₅ alkyl group, or an N-protective group;
(β) the di(α-D-mannopyranosyl-1) phosphates of formula II: 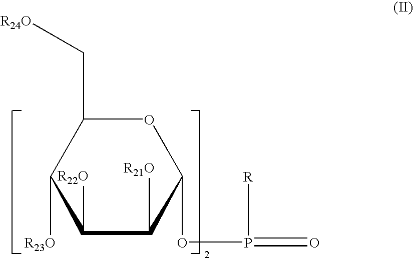
in which
R₂₁, R₂₂, R₂₃ and R₂₄, which are identical or different, each represent the hydrogen atom or an OH-protective group, and
R represents
an OH group,
a C₁-C₂₀ alkoxy group,
a C₆-C₁₀ aryloxy group capable of being substituted with one or more C₁-C₅ alkyl, C₁-C₅ alkoxy, halo, CF₃ and/or nitro groups,
an arylalkyleneoxy group, where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
a group having the structure:
—O—CH(Q)-O—CO-alkyl, or
—O—CH(Q)-O—CO—O-alkyl
where Q is H or CH₃, and the alkyl residue is C₁-C₅,
a group —O—CH₂—CH(OH)—CH₂OH, where the OH groups may be protected,
an OB residue, where B is an ethylenically unsaturated aliphatic C₂-C₂₁ residue containing a linear or branched hydrocarbon chain, or a cycloaliphatic C₅-C₂₁ residue, or
an amino acid group having the structure VIIa: 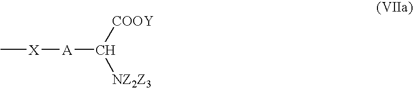
where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb: 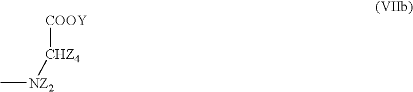
where
Y represents H or a C₂-C₅ alkyl group,
Z₄ is an alkylene, phenylene or phenylalkylene group, (where each alkylene group is C₁-C₅),
Z₂ represents H, a C₁-C₅ alkyl group, or an N-protective group;
(γ) the tri(α-D-mannopyranosyl-1) phosphates of formula III: 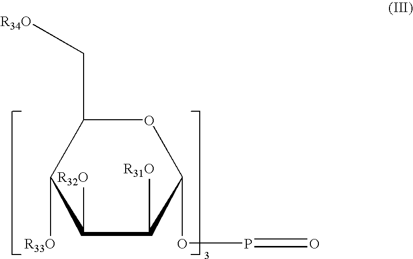
in which
R₃₁, R₃₂, R₃₃ and R₃₄, which are identical or different, each represent a hydrogen atom or an OH-protective group; and
(δ) mixtures thereof.

2. The composition as claimed in claim 1, for use as a medicament against the CDG-I syndrome.

3. The composition as claimed in claim 2, for use as a medicament against the CDG-Ia syndrome.

4. The composition as claimed in claim 1, wherein said OH-protective group for the hydroxyl groups at the 2-, 3-, 4- and 5-positions of the mannopyranosyl ring is an acyl group.

5. The composition as claimed in claim 1, wherein, in the formulae I, II and III, the OH-protective group for the OH functional groups at the 2-, 3-, 4- and 6-positions of the mannosyl ring is an aliphatic C₂-C₆ acyl group.

6. The use of a (mannosyl-1) phosphate derivative, said use being wherein use is made of a substance acting as an intracellular source of Man-1 P, which is chosen from the combination consisting of the compounds
(α) mono(α-D-mannopyranosyl-1) phosphates of formula I,
(β) di(α-D-mannopyranosyl-1) phosphates of formula II,
(γ) tri(α-D-mannopyranosyl-1) phosphates of formula III, and
(δ) mixtures thereof,
as claimed in claim 1,
for the preparation of a medicament intended for therapeutic use against the CDG-I syndrome, and in particular against the CDG-Ia syndrome.

7. A mannosyl-1 phosphate derivative, for use as a medicament, wherein it is chosen from the combination consisting of:
(α) mono(α-D-mannopyranosyl-1) phosphates of formula I: 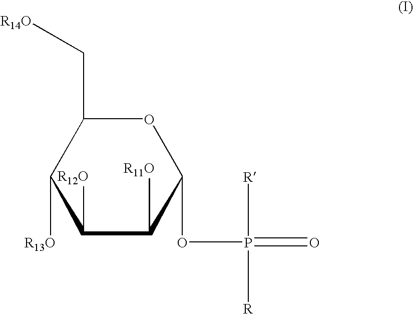
in which
R₁₁, R₁₂, R₁₃ and R₁₄, which are identical or different, each represent the hydrogen atom or an OH-protective group,
R and R′, which are identical or different, each represent
a C₆-C₁₀ aryloxy group (in particular phenoxy, 1-naphthyloxy or 2-naphthyloxy) capable of being substituted with one or more C₁-C₅ alkyl, C₁-C₅ alkoxy, halo, CF₃ and/or nitro groups,
an arylalkyleneoxy group (in particular OCH₂CH₂C₆H₅, OCH₂C₆H₅, 1-naphthylmethyloxy or 2-naphthylmethyloxy), where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
a group having a structure:
—O—CH(CH₃)—O—CO-alkyl, or
—O—CH(CH₃)—O—CO—O-alkyl
where the alkyl residue is C₁-C₅,
a group —O—CH₂—CH(OH)—CH₂OH, where the OH groups may be protected,
an OB residue, where B is an ethylenically unsaturated aliphatic C₂-C₂₁ residue containing a linear or branched hydrocarbon chain, or a C₅-C₂₁ cycloaliphatic residue, or
an amino acid group having the structure VIIa: 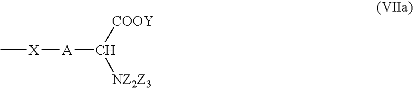
where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb: 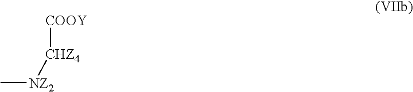
where
Y represents H or a C₂-C₅ alkyl group,
Z₄ is an alkylene, phenylene or phenylalkylene group, (where each alkylene group is C₁-C₅),
Z₂ represents H, a C₁-C₅ alkyl group, or an N-protective group;
(β) the di(α-D-mannopyranosyl-1) phosphates of formula II: 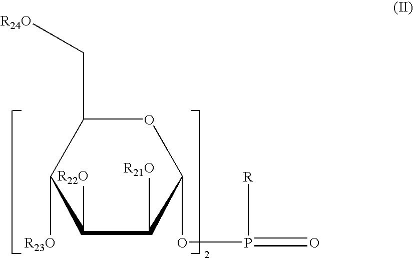
in which
R₂₁, R₂₂, R₂₃ and R₂₄, which are identical or different, each represent the hydrogen atom or an OH-protective group, and
R represents
an OH group,
a C₁-C₂₀ (preferably C₁-C₅) alkoxy group,
a C₆-C₁₀ aryloxy group capable of being substituted with one or more C₁-C₅ alkyl, C₁-C₅ alkoxy, halo, CF₃ and/or nitro groups,
an arylalkyleneoxy (in particular benzyloxy, phenylethyloxy, 1-naphthylmethyloxy or 2-naphthylmethyloxy) group, where the alkylene residue is C₁-C₅, and the aryl residue, which is C₆-C₁₀, is capable of being substituted with one or more C₁-C₅ alkyl, C₁-C₅ alkoxy, halo, CF₃ and/or nitro groups,
a group having the structure:
—O—CH(Q)-O—CO-alkyl, or
—O—CH(Q)-O—CO—O-alkyl
where Q is H or CH₃, and the alkyl residue is C₁-C₅,
a group —O—CH₂—CH(OH)—CH₂OH, where the OH groups may be protected,
an OB residue, where B is an ethylenically unsaturated aliphatic C₂-C₂₁ residue containing a linear or branched hydrocarbon chain, or a cycloaliphatic C₅-C₂₁ residue, or
an amino acid group having the structure VIIa: 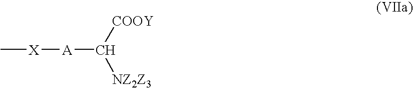
where
X is —O—, —S— or —NZ₁-,
Y represents H or a C₂-C₅ alkyl group,
A is an alkylene, phenylene or phenylalkylene group, (where each alkylene group is C₁-C₅),
Z₁ is H, a C₁-C₅ alkyl group or an N-protective group, and
Z₂ and Z₃, which are identical or different, each represent H, a C₁-C₅ alkyl group, or an N-protective group;
an amino acid group having the structure VIIb: 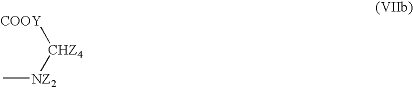
where
Y represents H or a C₂-C₅ alkyl group,
Z₄ is an alkylene, phenylene or phenylalkylene group, (where each alkylene group is C₁-C₅),
Z₂ represents H, a C₁-C₅ alkyl group, or an N-protective group;
(γ) the tri(α-D-mannopyranosyl-1) phosphates of formula III′: 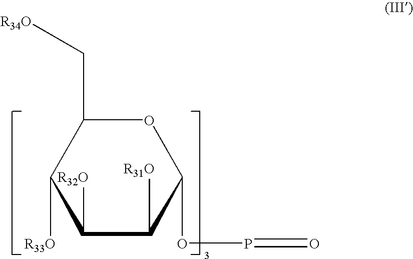
in which
R₃₁, R₃₂, R₃₃ and R₃₄, which are identical or different, each represent an OH-protective group having at least three carbon atoms; and
(δ) mixtures thereof.

8. The (α-D-mannosyl-1) phosphate derivative as claimed in claim 7, for use as a medicament against the CDG-I syndrome.

9. The (α-D-mannosyl-1) phosphate derivative as claimed in claim 8, for use as a medicament against the CDG-Ia syndrome.

10. The (α-D-mannosyl-1) phosphate derivative as claimed in claim 7, wherein, in the formulae I and II, the OH-protective group for the OH functional groups at the 2-, 3-, 4- and 6-positions of the mannosyl ring is an aliphatic C₂-C₆ acyl group, and in that, in the formula III′, the OH-protective group for the OH functional groups at the 2-, 3-, 4- and 6-positions of the mannosyl ring is an aliphatic C₃-C₆ acyl group.

11. The (α-D-mannosyl-1) phosphate derivative as claimed in claim 7, wherein the group R (or R′) is a group OB where B is an ethylenically unsaturated aliphatic C₂-C₂₁ residue, which may contain one or more double bonds C═C, containing a linear or branched hydrocarbon chain, or a cycloaliphatic C₅-C₂₁ residue, OB being in particular a group —O—CH₂—CH═C(CH₃)₂, O—(CH₂)₂—CH═C(CH₃)₂ or a terpeneoxy group.

12. The (α-D-mannosyl-1) phosphate derivative as claimed in claim 7, wherein the group R (or R′) is a group OB=terpeneoxy, in which the terpene portion is cyclic or acyclic, OB being in particular a farnesyloxy or geranyloxy group.

13. The (α-D-mannosyl-1) phosphate derivative as claimed in claim 7, wherein the group R (or R′) is a group having the structure VII obtained from an amino acid containing an amine or hydroxyl side functional group.

14. The (α-D-mannosyl-1) phosphate derivative as claimed in claim 7, wherein the group R (or R′) represents in the formula I or II:
(α) a phenoxy or 1-naphthyloxy group,
(β) a benzyloxy or 1-naphthylmethoxy group,
(γ) a group —O—CH(Q)-O—CO—O—(C₁-C₅)alkyl, where Q is H or CH₃,
(δ) a group —O—CH₂—CH(OH)—CH₂OH, where the OH groups may be protected,
(ε) a group εLys, pTyr; βSer or βThr, whose structures (where the NH₂ or COOH groups may be protected) are the following:
εLys: —NH—(CH₂)₄—CH(NH₂)COOH,
pTyr: -(p-O)—C₆H₄—CH₂-CH(NH₂)COOH,
βSer: —O—CH₂—CH(NH₂)COOH, and
βThr: —O—CH(CH₃)—CH(NH₂)COOH, and
(ξ) a group
—NH—(CH₂)₃—CH(NH₂)COOH or
—NH—(CH₂)₂—CH(NH₂)COOH,
where the NH₂ or COOH functional groups may be protected, it being also possible for R to represent in the formula II:
(η) a group —O—CH(Q)-O—CO—(C₁-C₅)alkyl, where Q is H or CH₃.

15. A method for preparing a compound of formula I, II or III′ as claimed in claim 7, wherein said method comprises
(a) the reaction of a 1-bromomannopyranose of formula (IVa): 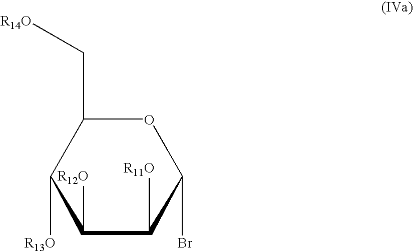
where R₁₁, R₁₂, R₁₃ and R₁₄ are defined as indicated above, with a monosilver phosphate of formula (Va): 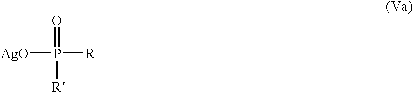
where R and R′ are defined as indicated above,
in order to obtain a mono(α-D-mannopyranosyl-1) phosphate compound of formula I;
(b) the reaction of a 1-bromomannopyranose of formula (IVb): 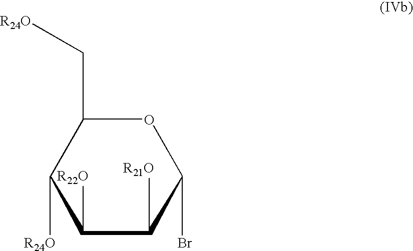
where R₂₁, R₂₂, R₂₃ and R₂₄ are defined as indicated above,
with a disilver phosphate of formula (Vb): 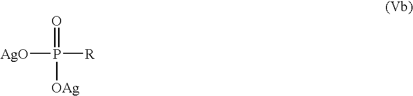
where R is defined as indicated above,
in order to obtain a di(α-D-mannopyranosyl-1) phosphate compound of formula II; or
(c) the reaction of a 1-bromomannopyranose of formula (IVc): 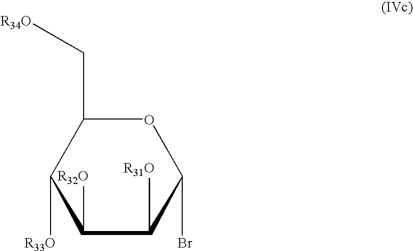
where R₃₁, R₃₂, R₃₃ and R₃₄, which are identical or different, each represent an OH-protective group which is a C₃-C₆ acyl group,
with a trisilver phosphate of formula (Vc): 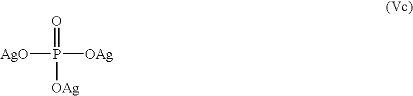
in order to obtain a tri(α-D-mannopyranosyl-1) phosphate compound of formula III′.

16. The method as claimed in claim 15, wherein the reaction of IVa with Va, the reaction of IVb with Vb or the reaction of IVc with Vc is carried out at a temperature of 15 to 40° C., preferably at room temperature (15-25° C.), advantageously in an appropriate inert solvent, preferably toluene, in the presence of a molecular sieve.

17. The method as claimed in claim 15, wherein the silver phosphate of formula Va, Vb or, respectively, Vc may be replaced by a phosphate of formula VIa, VIb or, respectively, VIc: 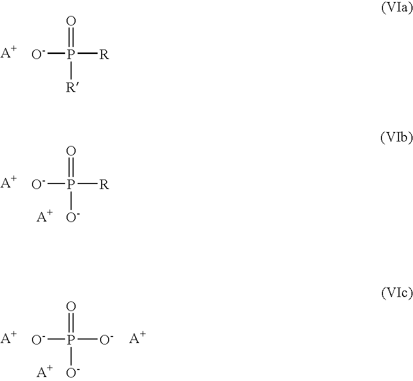
in which R and R′ are defined as indicated above, and A is H or R″₄N, R″ being H or an N-alkyl, cycloalkyl or aromatic group.
